(19) United States
(12) Patent Application Publication
(10) Pub. No.: US 2025/0384973 A1
(43) Pub. Date: Dec. 18, 2025

(54) METHOD FOR SELECTING QUESTIONS TO BE ANSWERED BY A PATIENT AND METHOD FOR CONDUCTING A PATIENT SURVEY

(71) Applicant: BIOTRONIK SE & Co. KG, Berlin (DE)

(72) Inventors: Steffen ZANDER, Berlin (DE); Henrik DEUTSCHMANN, Berlin (DE); Richard JORDAN, Berlin (DE)

(21) Appl. No.: 18/875,947

(22) PCT Filed: Jun. 15, 2023
(86) PCT No.: PCT/EP2023/066071

§ 371 Date: Dec. 17, 2024

(30) Foreign Application Priority Data
Jun. 22, 2022 (EP) 22180417.2

(51) Int. Cl. G16H 10/20

(52) U.S. Cl. CPC G16H 10/20

(57) ABSTRACT

A method for selecting questions to be answered by a patient includes receiving, from a patient database (3), patient data (5) indicative of a health condition of the patient. The patient data (5) includes sensor data (7) which has been generated by at least one sensor (8) for determining the health condition of the patient. The method further includes inputting the patient data (5) as input data (9) into a question selection algorithm (10) configured for selecting questions, based on the input data (9), from a list (6) of predetermined questions stored in a question database (4). The method further includes outputting at least one selected question or a list (13) of selected questions to be answered by the patient as output data (14) by the question selection algorithm (10). The patient data (5) includes at least one of anamnesis data (16), or medication data (19) of the patient.

The present invention relates to a computer-implemented method for selecting questions to be answered by a patient. Furthermore, the invention relates to a computer-implemented method for conducting a patient survey. In addition, the invention relates to a data processing device, a patient survey system and a computer program for carrying out one or both of these methods and to a computer-readable medium in which the computer program is stored.

Current automated patient surveys, which, for example, may be performed via websites, patient facing apps or voice bots, are mostly static, i.e., the set and number of questions is fixed and predetermined. Such patient surveys usually focus on very general questions and/or situations.

US patent application 2020/0105381 A1 discloses a computer-implemented method comprising: outputting questions to a user via one or more user devices, and receiving back responses to some of the questions from the user via one or more user devices; over time, controlling the outputting of the questions so as to output the questions under circumstances of different values for each of one or more items of metadata; monitoring whether or not the user responds when the question is output with the different metadata values; training the machine learning algorithm to learn a value of each of the items of metadata which optimizes a reward function, and based thereon selecting a time and/or location at which to output subsequent questions.

On the other hand, adapting questions of patient surveys to specific aspects of a single patient and/or to a specific patient journey may be very time-consuming, especially for large patient databases, since many different aspects have to be considered and appropriate questions have to be selected for each single aspect.

It is therefore an objective of the present invention to provide an improved method for selecting questions to be answered by a patient. Another objective of the invention is to provide an improved method for conducting a patient survey. Yet another objective of the invention is to provide a device, a system, a computer program and a computer-readable medium for carrying out one or both of these methods.

These objectives may be achieved by the subject-matter of the independent claims. Advantageous embodiments are defined in the dependent claims as well as in the corresponding specification and figures.

A first aspect of the invention relates to a computer-implemented method for selecting questions to be answered by a patient. The method comprises: receiving, from a patient database, patient data indicative of a health condition of the patient, the patient data at least comprising sensor data which has been generated by at least one sensor for determining the health condition of the patient; inputting the patient data as input data into a question selection algorithm configured for selecting questions, based on the input data, from a list of predetermined questions stored in a question database; and outputting at least one selected question or a list of selected questions to be answered by the patient as output data by the question selection algorithm. The method may be carried out automatically by a processor. The patient data may additionally comprise at least one of anamnesis data, diagnosis data or medication data of the patient.

In other words, the patient data may indicate results from one or more medical examinations of the patient. Using such medical data makes it possible to automatically select the questions in dependence of actual and/or potential health issues of the patient.

According to an embodiment of the present invention, the patient data may additionally comprise diagnosis data of the patient.

In addition to the sensor data, the patient data may comprise personal data or social of the patient, e.g., name, gender, age, profession or contact information, and/or medical data of the patient, e.g., results from one or more medical examinations (see below). Such medical data may also include data about implants the patient is carrying. The patient database may store patient data of different patients.

The sensor may be part of a stationary medical device. However, the sensor may also be part of a mobile device carried by the patient, e.g., a mobile medical device, smartphone, smartwatch, wearable (such as a fitness or sleep tracker), tablet or laptop. The sensor may be at least partially implanted in the patient's body, e.g., the patient's heart, brain, spine, ear or blood vessel. It is possible that the sensor data has been generated by different types of sensors. For example, the sensor may be an electrical/bioelectrical and/or optical and/or chemical/biochemical, in particular according to embodiments of the invention, an optical heart sensor, pulse oximeter, camera, thermometer, accelerometer, gyroscope, altimeter, barometer, GPS receiver or a combination of at least two of these examples. As mentioned above, the patient data may also comprise metadata for the sensor(s).

The list of selected questions may comprise significantly fewer items than the list of predetermined questions. For example, the list of predetermined questions may comprise more than 100, more than 1000, more than 10000 or even more than 100000 predetermined questions as items, whereas the list of selected questions may comprise no more than 500, no more than 100, no more than 50 or even no more than 10 selected questions as items.

The question selection algorithm may have been configured, e.g., trained, to classify the patient based on the input data and to generate the at least one selected question or the list of selected questions based on one or more classes associated with the patient. In this case, each class may be associated with one or more predetermined questions, or one or more predetermined lists of questions. Such classes may, for example, be different diseases and/or different types of diseases, different levels of physical and/or mental activity or different geographical and/or cultural regions.

For example, the at least one selected question or list of selected questions may be composed of one or more questions selected from each predetermined list associated with the class or classes of the patient. In a very simple case, the at least one selected question or list of selected questions may be composed of all the questions associated with the class or classes of the patient, or may be composed of all the questions figuring in each predetermined list associated with the class or classes of the patient.

A second aspect of the invention relates to a computer-implemented method for conducting a patient survey. The method comprises: generating a list of selected questions with the method for selecting questions as described above and below; sending the list of selected questions to a user device configured for presenting the selected questions to the patient and generating a list of answers by processing an input of the patient with respect to the selected questions; receiving the list of answers from the user device; and storing the list of answers to the patient database.

According to embodiments of the invention, the selected questions can be presented to a user in different formats, e.g. visual, audiovisual or audio. According to embodiments, the selected questions can be presented to the user via reading/writing, voice output, voice recognition and voicebot support.

The method may be carried out automatically by a processor.

The user device may, for example, be a telephone, smartphone, smartwatch, tablet, laptop or PC.

The list of answers sent to the patient database may be used to update the patient data of the respective patient. In other words, the list of answers may be used to select questions for a future survey with the same patient.

A third aspect of the invention relates to a data processing device comprising a processor configured for carrying out at least one of the methods as described above and below. The data processing device may include hardware and/or software modules. In addition to the processor, the data processing device may include a memory and data communication interfaces for data communication with peripheral devices. For example, the data processing device may be a server, PC, laptop, tablet or smartphone. It may be that at least one of the patient database or the question database is stored in the memory of the data processing device. Alternatively, the patient database and the question database may each be stored on the same or different external servers which are connected to the data processing device for data communication.

A fourth aspect of the invention relates to a patient survey system. The patient survey system comprises: a patient database which stores patient data of different patients; a question database which stores a list of predetermined questions; and the data processing device as described above and below.

Further aspects of the invention relate to a computer program comprising instructions which, when the program is executed by a processor, cause the processor to carry out at least one of the methods as described above and below and to a computer-readable medium in which the computer program is stored. The computer program may be executed by a processor of the data processing device.

The computer-readable medium may be a volatile or non-volatile data storage device. For example, the computer-readable medium may be a hard drive, USB (universal serial bus) storage device, RAM (random-access memory), ROM (read-only memory), EPROM (erasable programmable read-only memory) or flash memory. The computer-readable medium may also be a data communication network for downloading program code, such as the Internet or a data cloud.

It has to be noted that features of the methods as described above and below may be features of the computer program, the computer-readable medium, the data processing device and the patient survey system, and vice versa.

Embodiments of the invention may be considered, without limiting the invention, as being based on the ideas and findings described below.

The approach described above and below makes it possible to take multiple aspects of a patient or a patient journey into account when automatically selecting questions for a patient survey. Thus, highly personalized patient surveys can be created or conducted in a very efficient and versatile way.

As a result, different indications included in the patient data may lead to different question assemblies. If, for example, the patient is diagnosed with sleep apnea, the patient survey system may add questions about sleep behavior and/or wellness feelings in the morning to the survey.

The system may also analyze existing events from implants and add questions like “Yesterday in the afternoon, did you feel a dizziness or breathlessness/dyspnea?”; “Last week, the implant recognized an increasing or high body temperature. Have you been ill and contacted your general practitioner?”; “Yesterday at 2:12 pm until 2:17 μm, the implant detected abnormal heart events. Have you recognized them as well? Do you know; if there has been an external trigger for the events? How did you feel between 2:12 μm and 2:17 pm ?”

According to an embodiment of the present invention, the system takes into consideration patient data obtained from peripheral devices, as for instance from an implantable medical device, a sensor, or a wearable device. For instance, patient parameters as the respiration rate, mean heart rate, or mean heart rate at rest.

Taking activity parameters into account, the system may add questions like “Last week, you had 3 days with high activity, this week none. What is the reason for the decrease?”, “Your body weight has been stable for 2 years but now increased steadily during the last 3 months. Have you changed your nutrition? Do you want to have medical consultation about this?”, “Over the last couple of days, your sleep time has only an average of 4 h and the measured sleep quality is low. What happened?”, “Last week you walked in mean 8000 steps. The last three days your mean steps per day was only 5000. What is the reason?” to the survey.

The system may also take local weather information and/or local news into account when selecting questions, as, for example, consecutive heavy rainfalls can impact the patient's activity or wellness. Therefore, the system may add questions in this regard to the survey.

Also, results of one or more previous surveys and/or questionnaires with the patient and/or a specific group of patients with similar characteristics may be taken into account by the system. If, for example, a patient answers one or more specific questions always in the same manner, the system may consider the respective question(s) as being insignificant. In this case, the system may remove the question from the survey or replace it with at least one more relevant question. For example, in case a large group of patients answer a question always in the same manner, the question could be removed from the system, and not only from the survey.

Hence, the quality of the survey and the answers to it may be increased drastically. This means that patients can be approached in a more personalized way, which may result in a higher customer satisfaction.

According to an embodiment, the sensor data may have been generated by at least one sensor worn by the patient. For example, the sensor may be in direct contact with the patient's skin and/or may be at least partially implanted in the patient's body. The sensor may be part of a user device such as a dedicated medical device or a more generic mobile device, e.g., a smartphone, smartwatch, wearable (such as a fitness or sleep tracker), tablet or laptop. The user device may be connected to the data processing device for data communication. For example, the sensor data may be transmitted to the data processing device on a regular basis, e.g., once per hour, day, week or month. This ensures that the patient data always includes up-to-date sensor data. Thus, the accuracy of the method can be improved.

According to an embodiment, the sensor may be an implant. In particular, the implant may be a cardiac implant, e.g. a pacemaker, a heart monitor or a defibrillator, and/or neurostimulator. In other words, the sensor data may indicate a cardiac and/or neurological condition of the patient. Thus, using such sensor data makes it possible to automatically select questions in dependence of the patient's cardiac and/or neurological condition.

According to an embodiment, the sensor data may indicate at least one of a heart rate, an electrocardiogram, a movement, a body temperature, a blood pressure level, a blood oxygen saturation or a blood glucose level of the patient. For example, the patient's movement may be defined by a measured number of steps and/or a measured walking and/or riding distance. Additionally, the sensor data may indicate at least one of a heart rate variability, a brain activity, a body temperature, a respiratory rate or a sweat rate of the patient. Such parameters are known to be very accurate indicators for the patient's health condition.

According to an embodiment, the patient data may additionally comprise at least one list of answers of the patient to items of at least one previous list of selected questions, which has been previously output by the question selection algorithm. In other words, questions figuring in a current list of selected questions may be selected in dependence of previous answers of the (same) patient. Additionally, previous answers of at least one other patient may be analyzed by the question selection algorithm for generating the list of selected questions. This may further improve the accuracy of the method.

According to an embodiment, the patient data may additionally comprise a current location of the patient. The current location may be indicated by geographic coordinates which, for example, may have been determined with a position sensor, e.g., a GPS receiver or altimeter. Additionally or alternatively, the current location may be provided by an address, e.g. at least the city of residence, included in the patient data. The current location of the patient may be correlated in time with the sensor data, e.g., with a time at which the sensor data has been generated by the sensor(s). The current location of the patient may have a certain influence on the patient's health condition, e.g., when the patient is in a very hot or cold weather zone and/or at a very high place above sea level. Thus, it may be helpful to consider such geographical influences when selecting health-related questions for the patient.

According to an embodiment, the method for selecting questions may further comprise a step of retrieving weather data from a weather database based on the current location of the patient and a step of inputting, additionally, the weather data as the input data into the question selection algorithm. Additionally or alternatively, the method may comprise a step of retrieving news data from a news database based on the current location of the patient and a step of inputting, additionally, the news data as the input data into the question selection algorithm. As mentioned above, environmental conditions such as weather events or any other kind of events relating to the patient's location, such as political, economic or social events, may have a significant impact on the patient's health condition. This embodiment makes it possible to automatically select questions for the patient in dependence of such significant events. This helps to improve the accuracy of the method.

According to an embodiment, the question selection algorithm may have been trained, with different sets of exemplary input data and reference output data for each set of exemplary input data, to generate the output data from the input data. In other words, the question selection algorithm may comprise or consist of a machine learning algorithm which may have been trained to filter the most important questions according to the available patient data of a specific patient. The exemplary input data and reference output data may be seen as training data. For example, the machine learning algorithm may be an artificial neural network (e.g., a single-layer or multilayer perceptron, convolutional neural network, recurrent neural network or long short-term memory), a statistical method (e.g., a linear or logistic regression method or naive Bayes classifier), a support vector machine, a decision tree, a random forest or a combination of at least two of these examples. Using a trained question selection algorithm may significantly improve the accuracy of the method. This also makes it easier to modify the question selection algorithm, which may be done by retraining it with updated training data.

Generally, the trained question selection algorithm may be seen as a function with weights which have been adjusted automatically during training by an optimizer. The optimizer may be configured for minimizing a loss function which quantifies a difference between the reference output data and actual output data generated by the question selection algorithm from the exemplary input data. The optimizer may implement a variant of stochastic gradient descent which iteratively updates the weights by backpropagation. Alternatively, non-iterative methods may be used for computing the optimal weights.

Using an unsupervised training method for training the question selection algorithm is also possible.

The output of the question selection algorithm may be a Boolean value, e.g., “0” or “1”, or a probability, e.g., a percentage value between 0 and 1, for each item in a list of given classes, each class corresponding to a different selection of predetermined questions.

The questions used for training the question selection algorithm may comprise the same questions as those stored in the question database and/or questions that differ from those stored in the question database.

It is possible that the question selection algorithm has been trained to additionally modify the predetermined questions based on the input data and to output a list comprising at least one modified predetermined question as the output data.

Furthermore, the question selection algorithm may have been trained to additionally generate completely new questions from the predetermined questions and the input data and to output a list comprising at least one completely new question as the output data.

Each set of exemplary input data may comprise exemplary patient data, wherein the exemplary patient data may comprise exemplary sensor data. The exemplary sensor data may have been generated by the same sensor(s) as the one(s) used to generate the sensor data and/or by one or more sensors which differ from the one(s) used to generate the sensor data and/or by a simulated sensor, i.e., a mathematical model of the (real) sensor(s) used to generate the sensor data. In other words, the exemplary sensor data may be real and/or simulated data.

The reference output data may indicate a specific selection of predetermined questions for each set of exemplary input data.

It may be that the exemplary patient data additionally comprises at least one of exemplary anamnesis data, exemplary diagnosis data, exemplary indication data or exemplary medication data.

Additionally or alternatively, the exemplary patient data may comprise exemplary answers to some or all of the predetermined questions.

Additionally or alternatively, the exemplary patient data may comprise an exemplary location.

Additionally or alternatively, each set of exemplary input data may comprise at least one of exemplary weather data or exemplary news data.

According to an embodiment, the question selection algorithm is an artificial neural network.

The artificial neural network may have been trained as described above. The artificial neural network may, for example, be a single-layer or multilayer perceptron, convolutional neural network, recurrent neural network, long short-term memory or a combination of at least two of these examples. Alternatively, as pointed out above, the question selection algorithm may be a combination of the artificial neural network and at least one other type of machine learning algorithm.

It has to be noted that possible features and advantages of embodiments of the invention are described above and below partly with reference to a method for selecting questions and a method for conducting a patient survey, partly with reference to a corresponding data processing device and a corresponding patient survey system. A person skilled in the art will recognize that the features described for individual embodiments can be transferred, adapted and/or interchanged in an analogous and suitable manner to other embodiments in order to arrive at further embodiments of the invention and possibly synergistic effects.

Advantageous embodiments of the invention are further explained below with reference to the accompanying drawings. Neither the drawings nor the description are to be interpreted as limiting the invention.

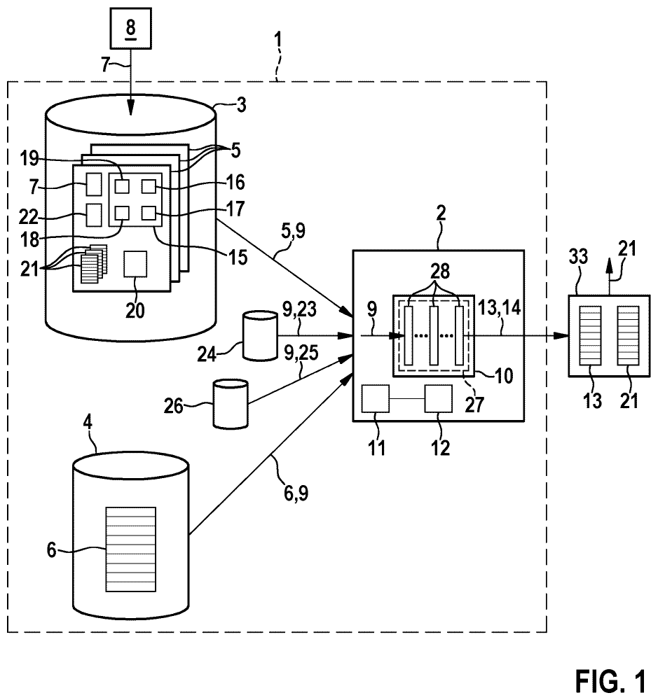

FIG. 1 shows a patient survey system according to an embodiment of the invention.

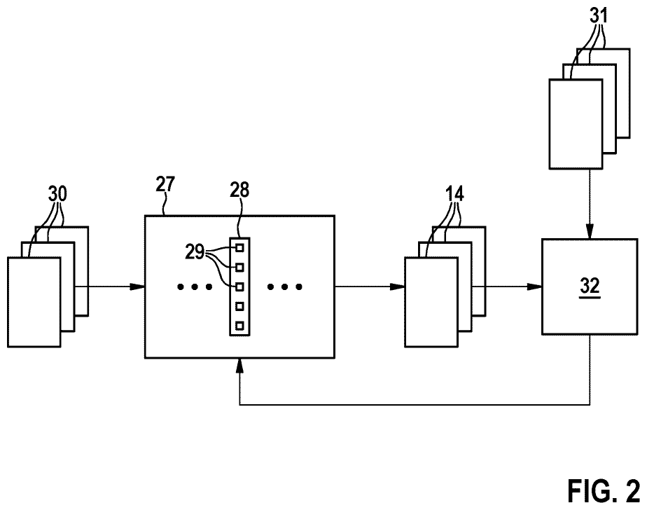

FIG. 2 illustrates a method for training an artificial neural network run by a processor of the patient survey system.

The figures are merely schematic and not to scale. Identical reference signs refer to identical or similar features.

FIG. 1 shows a patient survey system 1 comprising a data processing device 2, a patient database 3 and a question database 4.

The data processing device 2 receives patient data 5 from the patient database 3 in which sets of patient data 5 for different patients are stored. The patient data 5 indicates a health condition of the respective patient.

From the question database 4, the data processing device 2 further receives a list 6 of predetermined questions which may be asked to a patient.

The patient data 5 may be requested by the data processing device 2 on a regular basis, e.g., each time a set of patient data 7 is created and/or modified and/or in regular time intervals, e.g., once per hour, day, week and/or month.

The patient data 5 comprises sensor data 7 which has been generated by one or more sensors 8 adapted for determining the health condition of the respective patient.

The sensor 8 may be worn by the patient. It is possible that the sensor 8 is implanted in the patient's body. In particular, the sensor 8 may be (part of) an implant in the form of a cardiac pacemaker and/or neurostimulator, e.g., spinal cord stimulator.

Accordingly, the sensor data 7 may indicate at least one of a heart rate, an electrocardiogram or a neurological activity of the patient.

The sensor data 7 may also be provided by different types of sensors 8. For example, the sensor data 7 may additionally indicate at least one of a (walking) movement, a blood oxygen saturation or a blood glucose level of the patient.

The patient data 5 is input as input data 9 into a question selection algorithm 10 which is executed by a processor 11 of the data processing device 2.

The processor 11 may be connected to a memory 12 of the data processing device 2. A computer program may be stored in the memory 12, and the processor 11 may execute the question selection algorithm 10 by executing the internally stored computer program.

The question selection algorithm 10 analyzes the input data 9 and generates a list 13 of selected questions as output data 14 from the list 6 of predetermined questions. The list 6 of predetermined questions may be part of the input data 9.

The patient data 7 may comprise medical data 15 which further determines the health condition of the respective patient, such as, for example, anamnesis data 16 indicating an anamnesis of the patient, diagnosis data 17 indicating one or more diagnoses of the patient, indication data 18 indicating one or more medical indications of the patient and/or medication data 19 indicating one or more medications of the patient. This makes it possible to generate the list 13 of selected questions with respect to health issues and/or an entire patient journey of the patient.

The patient data 7 may comprise personal data 20 which identifies the respective patient, such as, for example, name, gender, birthdate, profession or contact information.

The patient data 7 may also comprise one or more lists 21 of answers of the respective patient to questions which have been previously selected for the respective patient by the question selection algorithm 10. Accordingly, the question selection algorithm 10 may generate the list 13 of selected questions with respect to the previous answers. This further improves the accuracy of the question selection algorithm 10.

Furthermore, the patient data 5 may comprise position data 22 indicative of a current location of the respective patient. The position data 22 may have been generated by a position sensor 8, e.g., a GPS receiver or altimeter. It is also possible that the position data 22 is derived from an address stored in the personal data 20. Accordingly, the question selection algorithm 10 may generate the list 13 of selected questions using, additionally, the position data 22.

For example, the question selection algorithm 10 may use the position data 22 to retrieve, e.g., over the Internet, weather data 23 from a weather database 24 and/or news data 25 from a news database 26, and may input, additionally, the weather data 23 and/or the news data 25 as the input data 9 into the question selection algorithm 10. Accordingly, the question selection algorithm 10 may generate the list 13 of selected questions additionally based on the weather data 23 and/or the news data 25.

The question selection algorithm 10 may comprise at least one artificial neural network 27 composed of one or more layers 28 with trainable weights 29 (see FIG. 2). However, it is also possible to implement the question selection algorithm 10 using other machine learning models or no machine learning model at all.

As shown in FIG. 2, the artificial neural network 27 may have been trained with different sets of exemplary input data 30 and different sets of reference output data 31, wherein each set of reference output data 31 may be linked to one of the sets of exemplary input data 30.

During training, the artificial neural network 27 may generate (actual) output data 14 from the exemplary input data 30. An optimizer 32 may then compare the output data 14 to the corresponding reference output data 31 using an appropriate loss function and minimize the loss function by iteratively modifying the weights 29 with a stochastic gradient descent method.

In a further step, the list 13 of selected questions may be sent from the data processing device 2 to a user device 33 such as a telephone, smartphone, smartwatch, tablet, laptop or

PC. The user device 33 may then display and/or read the selected questions to the respective patient and generate a list 21 of answers based on an input of the patient.

Next, the user device 33 may send the list 21 of answers to the patient survey system 1, e.g., to the data processing device 2 and/or the patient database 3, which may update the corresponding patient data 7 accordingly. The updated patient data 7 may then be input as the input data 9 in the question selection algorithm 10 to generate an updated list 13 of selected questions which may be used in a future survey.

As shown in FIG. 1, the patient survey system 1 may be realized in a distributed computer environment. In this case, the patient database 3 and the question database 4 may be stored on one or more external servers connected to the data processing device 2 for data communication, e.g., over the Internet.

Alternatively, one or both of the databases 3, 4 may be stored in the memory 12 of the data processing device 2.

The question database 4 may be a relatively simple database containing questions for a variety of usage scenarios, e.g., questions relating to different indications, optionally including existing standard catalogues, conversational questions in order to extend the questionnaire to a real conversation, situational well-being questions covering different activities, weather, etc., or situational health questions relating to post-surgery and/or long-time care.

The question selection process is performed by executing the question selection algorithm 10. During the question selection process, at least the patient data 5 are analyzed and corresponding questions are selected automatically. For example, the question selection algorithm 10, knowing the indication of the patient, can select indication-based questions from the list 6 of predetermined questions.

As mentioned above, the question selection algorithm 10 may be configured for analyzing an answer behavior of the patient. In this case, the question selection algorithm 10 may determine the best option for contacting the patient in dependence of the answer behavior.

It is possible that the question selection algorithm 10 determines similarities between different patients for selecting the most appropriate questions.

The question selection process may comprise at least one of the following steps: analyzing indications and selecting respective questions from a list of indication-related questions; analyzing activities, e.g., number of steps and/or walking distance, and selecting respective questions from a list of activity-related questions; analyzing implant data, including implant alarms, and/or trends of such data and selecting respective questions from a list of implant-related questions; or analyzing social data such as the patient's birthday or birthdays of family members and selecting respective questions from a list of birthday-related questions (for example, on such events, questions for a very short “well-being on your birthday” survey may be selected). As a next step, the data processing device 2 may analyze available data about contact options for the respective patient, i.e., the personal data 20.

It has to be noted that, in the claims, the word “comprising” does not exclude other elements or steps, and the indefinite article “a” or “an” does not exclude a plurality. A single processor or controller or other unit may fulfil the functions of several items recited in the claims. The mere fact that certain measures are recited in mutually different dependent claims does not indicate that a combination of these measures cannot be used to advantage. Any reference signs in the claims should not be construed as limiting the scope of the claims.

### LIST OF REFERENCE SIGNS

1 patient survey system
2 data processing device
3 patient database
4 question database
5 patient data
6 list of predetermined questions
7 sensor data
8 sensor
9 input data
10 question selection algorithm
11 processor
12 memory
13 list of selected questions
14 output data
15 medical data
16 anamnesis data
17 diagnosis data
18 indication data
19 medication data
20 personal data
21 list of answers
22 position data
23 weather data
24 weather database
25 news data
26 news database
27 artificial neural network
28 layer
29 weight
30 exemplary input data
31 reference output data
32 optimizer
33 user device

1. A computer-implemented method for selecting questions to be answered by a patient, the method comprising:
receiving, from a patient database (3), patient data (5) indicative of a health condition of the patient, the patient data (5) comprising sensor data (7) which has been generated by at least one sensor (8) for determining the health condition of the patient;
inputting the patient data (5) as input data (9) into a question selection algorithm (10) configured for selecting questions, based on the input data (9), from a list (6) of predetermined questions stored in a question database (4); and
outputting at least one selected question or a list (13) of selected questions to be answered by the patient as output data (14) by the question selection algorithm (10),
wherein the patient data (5) additionally comprises at least one of anamnesis data (16), diagnosis data (17), indication data (18) or medication data (19) of the patient.

2. The method of claim 1,
wherein the sensor data (7) has been generated by at least one sensor (8) worn by the patient.

3. The method of claim 2,
wherein the sensor (8) is either an implantable sensor or part of an implantable medical device.

4. The method of claim 1,
wherein the sensor data (7) indicates at least one of a heart rate, an electrocardiogram, a movement, a body temperature, a blood pressure level, a blood oxygen saturation, or a blood glucose level of the patient.

5. The method of claim 1,
wherein the patient data (5) additionally comprises at least one answer or at least one list (21) of answers of the patient to items of at least one previous selected question or list (13) of selected questions, which has been previously output by the question selection algorithm (10).

6. The method of claim 1,
wherein the patient data (5) additionally comprises a current location of the patient.

7. The method of claim 6, further comprising one or both of:
retrieving weather data (23) from a weather database (24) based on the current location of the patient and inputting, additionally, the weather data (23) as the input data (9) into the question selection algorithm (10); or
retrieving news data (25) from a news database (26) based on the current location of the patient and inputting, additionally, the news data (25) as the input data (9) into the question selection algorithm (10).

8. The method of claim 1,
wherein the question selection algorithm (10) has been trained, with different sets of exemplary input data (30) and reference output data (31) for each set of exemplary input data (30), to generate the output data (14) from the input data (9).

9. The method of claim 8,
wherein the question selection algorithm (10) is an artificial neural network (27).

10. A computer-implemented method for conducting a patient survey, the method comprising:
generating a list (13) of selected questions with the method of claim 1;
sending the list (13) of selected questions to a user device (33) configured for presenting the selected questions to the patient and generating a list (21) of answers by:
processing an input of the patient with respect to the selected questions;
receiving the list (21) of answers from the user device (33); and
storing the list (21) of answers in the patient database (3).

11. A data processing device (2) comprising a processor (11) configured for carrying out the method of claim 11.

12. A patient survey system (1), comprising:
a patient database (3) which stores patient data (5) of different patients;
a question database (4) which stores a list (6) of predetermined questions; and
the data processing device (2) of claim 12.

13. A computer program comprising instructions which, when the program is executed by a processor (11), cause the processor (11) to carry out the method of claim 11.

14. A computer-readable medium comprising instructions which, when executed by a processor (11), cause the processor (11) to carry out the method of claim 11.
